(19) United States
(12) Patent Application Publication
(10) Pub. No.: US 2008/0161745 A1
(43) Pub. Date: Jul. 3, 2008

(54) Bleaching of contrast enhancing agent applied to skin for use with a dermatological treatment system

(76) Inventor: Oliver Stumpp, Palo Alto, CA (US)

Correspondence Address: RELIANT / FENWICK;c/o FENWICK & WEST, LLP, 801 CALIFORNIA STREET, MOUNTAIN VIEW, CA, 94041, US

(21) Appl. No.: 11/899,771

(22) Filed: Sep. 7, 2007

Related U.S. Application Data
(60) Provisional application No. 60/843,096, filed on Sep. 8, 2006.

(51) Int. Cl. A61K 8/18; A61Q 19/02; A61N 1/30

(52) U.S. Cl. 604/20; 424/62

(57) ABSTRACT

A contrast enhancing agent can be applied to the skin to enhance or enable the response of a sensor element in a dermatological handpiece for measurement of one or more positional parameters of the handpiece. The positional parameter(s) can be used as part of a feedback system that controls the treatment dosage. A bleaching agent is applied to the skin surface to reduce the visibility of the topically applied contrast enhancing agent as part of a treatment with a dermatological handpiece wherein at least one positional parameter of the handpiece is measured and used to control the treatment dosage.

### CROSS-REFERENCE TO RELATED APPLICATIONS

This application claims priority under 35 U.S.C. § 119(e) to U.S. Provisional Patent Application Ser. No. 60/843,096, “Bleaching of contrast enhancing agent applied to skin for use with a dermatological treatment system,” filed Sep. 8, 2006. This application relates to U.S. patent application Ser. No. 10/745,761, “Method and apparatus for monitoring and controlling laser-induced tissue treatment,” by Leonard C. DeBenedictis and Thomas R. Myers, filed Dec. 23, 2003; to U.S. patent application Ser. No. 11/020,648, “Method and apparatus for monitoring and controlling laser-induced tissue treatment,” by Len DeBenedictis, Tom Myers, George Frangineas, Kin F. Chan, filed Dec. 21, 2004; and to U.S. Patent Application Ser. No. 60/712,358, “Method and Apparatus for Monitoring and Controlling Thermally Induced Tissue Treatment,” by Leonard C. DeBenedictis, George Frangineas, Kin F. Chan, B. Wayne Stuart III, Robert Kehl Sink, Thomas R. Myers and Basil Hantash, filed Aug. 29, 2005. The subject matter of all of the foregoing is incorporated herein by reference in their entirety.

### BACKGROUND OF THE INVENTION

1. Field of the Invention

This invention relates generally to bleaching a contrast enhancing agent that is applied to the skin. More particularly, it relates to bleaching a topically applied contrast enhancing agent following an electromagnetic treatment in which a treatment sensor uses the contrast enhancing agent as part of a feedback system.

2. Description of the Related Art

In dermatological electromagnetic treatment systems, the treatment dosage can be controlled to reduce the incidence of over- and under-treatment. For example, the electromagnetic dose delivered by a handpiece can be controlled within a desired range by using feedback to adjust the timing of treatment pulses in response to changes in relative position or speed of the handpiece across the skin.

Several such feedback systems have been proposed. For example, Weckwerth et al describe (in U.S. Pat. No. 6,758,845) a system which senses regularly spaced indicia that are drawn on the skin and automatically fires the laser after a selected number of indicia are detected. In another example, Debenedictis et al. describe (in co-pending U.S. patent application Ser. No. 11/020,648) topically applying a contrast-enhancing substance to the skin to improve the signal of an optical mouse chip that can measure handpiece velocity and adjust pulse repetition frequency. Both Weckwerth et al. and DeBenedictis et al. describe the application of a contrast enhancing agent, such as FD&C Blue #1, to the surface of the skin as part of a treatment. Following treatment, the contrast enhancing agent is typically removed by scrubbing with soap and water. This scrubbing irritates the skin due to harsh abrasive scrubbing on a skin surface that is sensitive due to the electromagnetic treatment. In addition, the scrubbing is not completely effective for removing the contrast enhancing agent, particularly for dye that has been absorbed in oily pores and active acnes. Residual contrast enhancing agent can create an unattractive appearance, particularly in cases where the contrast enhancing agent is used on the face.

Thus, there is a need for a less abrasive method to reduce the visibility of topically applied contrast enhancing agent that is applied for use with feedback systems in dermatological treatments.

### SUMMARY OF THE INVENTION

The present invention overcomes limitations of the prior art by applying a bleaching agent to the skin surface to reduce the optical absorption of the contrast enhancing agent in the visible spectrum of a topically-applied contrast enhancing agent that is applied to the skin for a dermatological treatment. The contrast enhancing agent can be applied to enhance the measurement by an optical sensor, such as an optical mouse chip sensor, a CMOS detector array, or a CCD camera. The optical sensor can be used to measure the positional parameters of a dermatological treatment handpiece during treatment of the selected region of skin.

In some embodiments, the contrast enhancing agent is applied topically. The contrast enhancing agent can be disposed in lines and/or patterns on the selected region, can be disposed in equally spaced indicia across the selected region, and/or can be applied in a substantially uniform concentration that selectively decorates at least some wrinkles and pores.

In some embodiments, the contrast enhancing agent comprises a triphenyl methane compound. In some embodiments, the contrast enhancing agent comprises at least one of methyl blue, water blue, aniline blue, royal blue, and basic blue 15. In some embodiments, the contrast enhancing agent comprises at least one of basic blue 8, basic blue 20, basic blue 26, fuchsin, crystal violet, eosin, and phenolphthalein.

In some embodiments, the bleaching agent comprises at least one of a peroxygen compound, a hypochlorite, a reducing agent, and chlorine dioxide. For example, the bleaching agent may comprise a peroxygen compound with a concentration in the range of about 3% by mass to about 20% by mass, a hypochlorite with a concentration in the range of about 0.6% by mass to about 3% by mass, a reducing agent with a concentration in the range of about 0.6% by mass to about 3% by mass, chlorine dioxide with a concentration in the range of about 0.5% by mass to about 5% by mass, or combinations thereof.

In some embodiments, the pH of the bleaching agent is between about 8.0 and about 12.0 or between about 9.0 and about 11.0.

In some embodiments, the bleaching agent further comprises an accelerating agent. For example, acetyl ethylene diamine can be used as an accelerating agent with some bleaching agents, particularly when used with a peroxygen compound as the bleaching agent. In some embodiments, the combination of acetyl ethylene diamine and a peroxygen compound can be used to bleach the color from FD&C Blue #1 that is used as a contrast enhancing agent.

In some embodiments, a bleaching agent can be dispensed during treatment as an indicator of the region that has been treated already. In some of these embodiments, the bleaching agent is part of a disposable tip. The bleaching agent can be dispensed directly from the tip.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The invention has other advantages and features which will be more readily apparent from the following detailed description of the invention and the appended claims, when taken in conjunction with the accompanying drawings, in which:

FIG. 1 is a diagram of an apparatus that can be used to practice the inventive method.

FIG. 2 is a diagram of an apparatus that can be used to practice the inventive method.

FIG. 3 is a block diagram of an embodiment of the inventive method.

FIGS. 4A-E is a series of pictures showing the results of the application of a bleaching agent to a contrast enhancing agent in a Petri dish.

FIGS. 5A-G is a series of pictures showing the results of the application of a bleaching agent to a contrast enhancing agent on human skin.

### DETAILED DESCRIPTION OF THE PREFERRED EMBODIMENTS

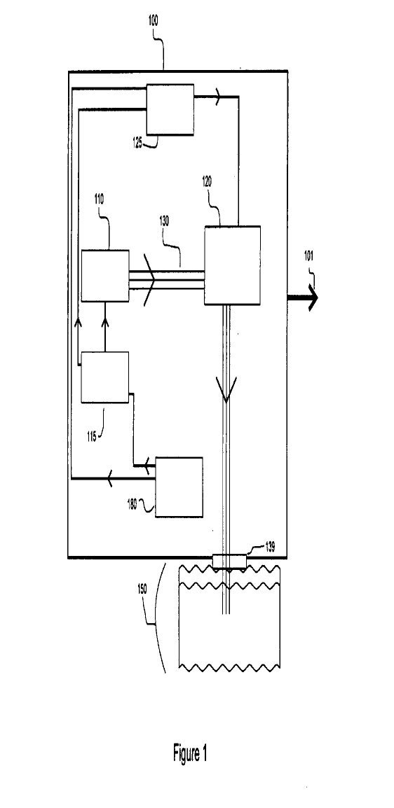

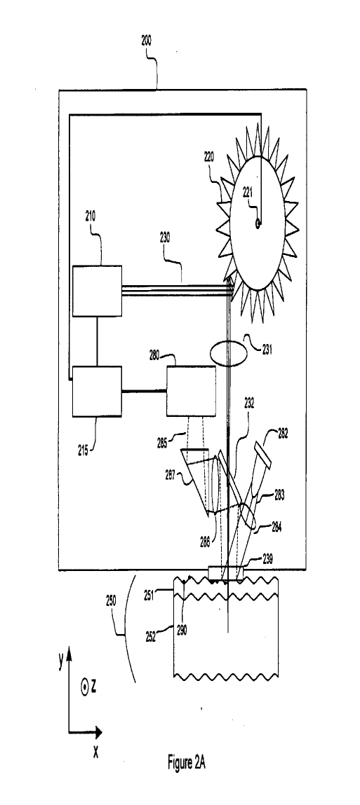

FIG. 1 is a diagram of an embodiment of the invention showing a manually movable handpiece 100 that is configured to deliver electromagnetic treatment energy to the skin 150 in the treatment region. The electromagnetic source 110 generates electromagnetic energy 130 that treats the skin. The controller 115 activates or adjusts one or more parameters of the electromagnetic source for the purpose of affecting treatment. The handpiece 100 may contain a controller 115 that may comprise a computer, a radio frequency generator, and/or laser driver electronics. In other configurations, the controller 115 is located external to the handpiece 100 and is operably connected to the handpiece 100 to control treatment parameters. The system may also include an optional scanning delivery unit 120 that is operably coupled to a scanner control 125 that scans the electromagnetic energy 130 over the treatment region of the skin 150. An optional contact plate 139 that is mechanically coupled to the handpiece 100 may be used to make good electrical or optical contact with the skin 150 to enhance controlled delivery of the electromagnetic energy 130. A positional sensor 180 measures positional parameters of the handpiece and a dosage evaluation sensor 160 measures skin response to treatment.

While the operator manually moves the handpiece 100 in direction 101 or after the operator has manually moved the handpiece 100, the positional sensor 180 measures one or more positional parameters of the handpiece 100. The positional sensor 180 communicates with the controller 115 and/or with the scanner control 125. The controller 115 and/or the scanner control 125 materially alter the treatment in real time in response to the positional parameter measurements and/or in response to the dosage evaluation measurements.

In some embodiments, the treatment system may further comprise a dosage evaluation sensor (not pictured) that allows additional capabilities as described in co-pending U.S. Patent Application No. 60/712,358, entitled “Method and Apparatus for Monitoring and Controlling Thermally Induced Tissue Treatment”, which is herein incorporated by reference.

In one embodiment of the invention, one or more measured handpiece positional parameters include handpiece position or handpiece angle (angular orientation) or the time derivatives of these two parameters including handpiece velocity, handpiece acceleration, handpiece angular velocity, and handpiece angular acceleration. Handpiece positional parameters can be absolute or can be relative to the treatment region.

The scanning delivery unit is configured to receive the electromagnetic energy 130 and deliver the electromagnetic energy 130 to the skin 150 regardless of where the other components are housed. For example, the electromagnetic source 110 may be a laser. The electromagnetic radiation may be coupled into an optical fiber, optical waveguide, or articulating arm for delivery to the handpiece. The handpiece can accept optical energy by using a fiber coupling or a fiber collimator. Similarly, it will be evident to those skilled in the art that the sensor 180 should be operably coupled to the controller 115, but do not need to be located inside the handpiece.

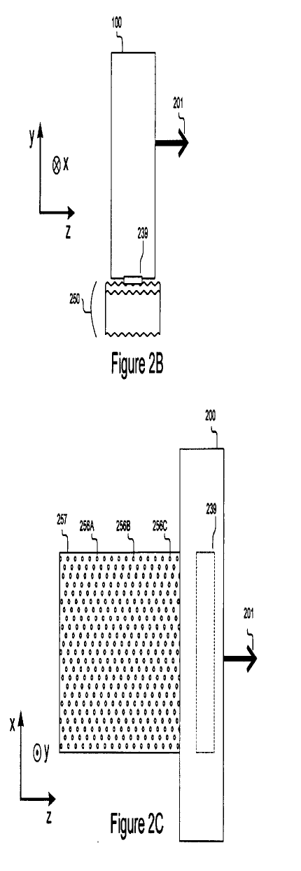

The controller 115 and scanner control 125 may be separate components as in FIG. 1 or may be combined as a single controller as shown in FIG. 2A.

In the embodiment of FIG. 2A, a laser source 210 is used as the electromagnetic source. In this embodiment, a manually movable handpiece 200 is configured to deliver an optical beam 230 of electromagnetic energy to the treatment region of the skin 250. The handpiece 200 contains a controller 215 comprising a computer and/or laser driver electronics. The controller 215 controls an optical source 210 and a scanning delivery unit 220 to affect one or more parameters such that treatment is materially affected. The optical source 210 generates an optical beam 230 that is directed to an optional scanning delivery unit 220. The scanning delivery unit 220 deflects the laser beam 230 to different treatment zones on or within the skin 250 as will be described in greater detail below. For clarity, only one beam position is shown in FIG. 2A. A dichroic mirror 232 and a contact plate 239 that are substantially transparent at the wavelength of the laser beam 230 may advantageously be included in particular embodiments. The deflected laser beam 230 is delivered through the dichroic mirror 232 and contact plate 239 to the skin 250. A beam delivery lens 231 can be used to focus the deflected beam 230 within the epidermis 251, dermis 252, or other layers of the skin 250. The focal point of the optical beam 230 may be below the skin surface or the beam may be diverging or collimated as it enters the skin 250.

In the embodiment of FIG. 2, the positional sensor 280 measures the position of the handpiece relative to the surface of the skin 250. In alternate embodiments, the positional sensor 280 could measure position, velocity, and/or acceleration of handpiece relative to the surface of the skin 250. An illumination source 282 emits illumination 283 that is collimated by an illumination delivery lens 284 for delivery to the surface of the skin 250. Collimating the illumination 283 increases alignment tolerances, improves uniformity of the illumination on the skin surface, and allows the illumination source 282 to be placed further from the treatment region than would otherwise produce a uniform profile of illumination 283 at the surface of the skin 250. The illumination 283 is scattered from the surface of the skin 250 or from a contrast enhancing agent 290 that is placed into or onto the skin 250. The spectral reflectivity of the dichroic mirror 232 and the reflective prism 287 are designed to substantially reflect the wavelength of the scattered illumination 285. A detector lens 286 is placed in the optical path from the skin to the positional sensor 280 to image the surface of the skin 250 on the optical positional sensor 280. Examples of optical positional sensors 280 include an optical mouse chip (Agilent Technologies, Palo Alto, Calif.), a CCD camera, a CMOS detector array, or an optical sensor array of at least two sensor elements. Preferably the optical sensor array has at least 25 sensor elements, arranged as a 5×5 array to have sufficient resolution to accurately quantify a range of velocity resolutions easily. Preferably, this optical positional sensor is silicon-based so that it can be manufactured cheaply using bulk manufacturing processes and cheap material sources that have been developed for the electronics industry. Other configurations will be evident to those skilled in the art.

In FIG. 2A, the direction 201 of handpiece motion (z direction) is essentially perpendicular to the plane of the page. FIG. 2B illustrates a side view of the handpiece that shows the direction of motion 201 of the handpiece 200. For simplicity, internal elements of the handpiece 200 are not shown in FIG. 2B. The handpiece 200 is manually moved by the operator in direction 201 while the positional sensor 280 measures one or more positional parameters of the handpiece and communicates with the controller 215. In response to the measurements, the controller 215 adjusts the optical treatment parameters in real time to materially affect the photothermal treatment. For example, the rate of laser firing can be adjusted to be proportion to the velocity of the handpiece 200 to create a predefined treatment pattern or a uniform treatment.

FIG. 2C shows a treatment pattern comprising separated microscopic treatment zones 256 that can be created with this approach as the handpiece 200 is moved across the treatment region 257 in the direction 201. In this embodiment, separated microscopic treatment zones 256A, 256B, and 256C can be created in the skin as described in co-pending U.S. application Ser. Nos. 10/367,582, 10/751,041, 10/888,356, and 60/652,891, which are herein incorporated by reference. Preferably, the treatment zones 256 are created in a predefined pattern that is invariant with the relative velocity or acceleration of the handpiece 100. Other patterns will be evident to those skilled in the art. Substantially uniform treatment coverage can be created by appropriately choosing optics, treatment parameters, and laser pulse timing.

In an alternative embodiment, the pattern can be intentionally varied according to a predefined algorithm where treatment rate is varied in real time in response to changes in the velocity or acceleration of the handpiece and where the treatment pattern is not predefined. For example, the treatment pattern can be controlled in real time by the user by appropriately adjusting the position, velocity, or acceleration of the handpiece. In some treatments, it is desirable to allow the operator to have control over the level of treatment through the use of velocity. For example, if the user treats quickly, this may be set up to allow a higher level of treatment. If the user treats slowly, then the maximum allowable treatment can be reduced. Thus, the user is able to control the treatment settings simply by changing positional parameters of the handpiece. Thus, the treatment pattern, treatment density, treatment intensity, and other treatment parameters may not be predefined, but may be defined through an automated response to measured positional parameters. An electronic or computer interface (not pictured) may be provided to allow switching on or off different modes of user control.

In another embodiment, a treatment status map is displayed on a monitor (not shown) for the user or the patient to observe. The positional sensor 280 can be used to measure the location within the treatment region of the tissue. In this way, a map can display which parts of the treatment region have been treated and how each part of the treatment region has responded to treatment. The user can take the information on this map to make treatment uniform over the entire treatment region or to have treatment vary in a desirable manner such as treating area with deep wrinkles more heavily than less wrinkled areas. Alternatively, the system can be configured to automatically reduce or disable treatment in the regions that have already been adequately treated as the user continues to move the handpiece over the treatment region. A picture or schematic representation of the treatment region, such as line drawing of a face for treatment of wrinkles on the face, can be used as a background for a computer display of the map of the treatment response measurements.

Controller 215, optical source 210, and other components may be external to the handpiece 200 instead of being included inside the handpiece as illustrated in FIG. 2A. The optical beam 230 can propagate to the handpiece through free space, through an articulated arm, or through a waveguide, such as an optical fiber. The handpiece 200 may be mechanically separable from or mechanically separate from the external components and the handpiece 200 may be configured to receive the optical beam 230 and/or the signal from the controller 215.

In a preferred embodiment, the electromagnetic source 210 is a single mode pulsed erbium doped fiber laser with a peak output power in the range of 5-50 W and a wavelength in the range of 1.52-1.62 μm. This laser source can be focused to an optical spot size in the range of 30-600 μm and preferably 60-300 μm on the surface of the skin. Pulse energies in the range 2-100 mJ and preferably in the range of 8-20 mJ can be used for these ranges of optical spot size, wavelength, and power. This preferred embodiment does not include surface skin cooling, but such cooling can be included if desired to reduce damage to the epidermis and dermal-epidermal junction.

The scanning delivery unit 220 used in this embodiment is a scanner wheel rotating at least 360° around an axis 221 as described in detail in co-pending U.S. Application No. 60/652,891, which is incorporated by reference herein. Other scanner types will be apparent to those skilled in the art. For example, galvanometer scanners, pseudo stationary deflection (PSD) scanners as described in co-pending U.S. application Ser. No. 10/750,790, which is also incorporated by reference herein, polygonal scanners, light valves, LCD screens, MEMS based reflective scanners, and translation stages can be used for the scanning delivery unit for delivery of optical energy. Multiple scanning delivery units can be used in such systems to control multiple axes of deflection.

One algorithm that can be used to control operational parameters of the scanning delivery unit 220 is to adjust the rotational speed of a double or single wheel PSD scanner and the laser firing rate in proportion to the velocity of the handpiece. This allows microscopic treatment zones of fractional resurfacing to be placed in a predefined pattern on the skin.

Another algorithm for controlling treatment is to adjust the firing of the laser in approximate proportion to the relative velocity of the handpiece to create a predefined density of treatment zones. A uniform distribution of treatment zones across a treatment region by overlapping or abutting treatment zones can also be achieved. For example, if the scanner 220 shown in FIG. 2A is controlled to spin at a constant angular velocity as the handpiece 200 is moving across the surface of the skin 250, the laser firing can be pulsed to create the desired density of treatment zones within the treatment region by firing the laser only when it is aligned with a particular facet of the scanner that creates the desired distribution or density of treatment. Not every facet needs to be used. For a particular velocity, every facet may be used. If the velocity is reduced by a factor of three from this velocity, then only every third facet can be used to keep the same density. Preferably, the algorithm maintains a uniform distribution of treatment zones within the treatment region. Spinning the scanning wheel 220 at a constant angular velocity is preferable to requiring the angular velocity of the scanning wheel 220 to be proportional to the speed of the handpiece 200 because this configuration reduces the complexity of the motors, associated drive electronics, and encoders that are used to accurately control the angular velocity of the scanning wheel 220.

To enhance the ability of the optical positional sensor 280 to read the positional parameters of the handpiece 200, a contrast enhancing agent 290 can be applied onto or into the skin 250. For example, uniform application of a dye to the surface of the skin 250 can preferentially decorate certain features, such as skin wrinkles or hair follicles, to create shapes that can be detected as objects by the positional sensor 280. The contrast enhancing agent 290 must be non-toxic when applied onto or into a patient's skin in amounts suitable for adequately enhancing measurements by the positional sensor 280. Preferably, the contrast enhancing agent and the materials and geometry chosen for the handpiece 200 and contact window 239 allow the handpiece 200 to slide easily over the surface of the skin 250.

Examples of contrast enhancing agents 290 are carbon particles, India ink, and FD&C Blue #1. Many other dyes, inks, particulates, etc. can be used as contrast enhancing agents when applied to the skin and when used with the appropriate positional sensor 280. The wavelength illumination source 282 can be chosen to maximize the signal to noise ratio of the measurement of the positional parameters of the handpiece 200. For example, a red LED with a peak wavelength in the range of 600 to 640 nm can be used with FD&C Blue #1.

In many cases, the contrast enhancing agent will be chosen such that it has a low absorption of the treatment energy or of the treatment wavelength in the case of optical treatment energy. In this way, the contrast enhancing agent will not interfere with the deposition of the treatment energy in the treatment region. In some cases, the contrast enhancing agent is chosen such that a measurable or observable parameter changes in response to the treatment energy. A change in the contrast enhancing agent can be used to determine where treatment has occurred, which allows the treatment to be touched up in areas where it is not even or uniform.

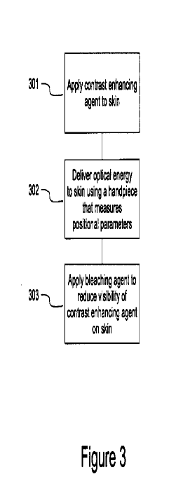

The contrast enhancing agent can also be applied in a pattern. The pattern may comprise a uniform grid of identical figures 391 in the treatment region 357 as illustrated in FIG. 3A. The pattern may comprise a nonuniform pattern of identical figures 392 in the treatment region 357 as illustrated in FIG. 3B. The pattern may comprise a nonuniform pattern of a plurality of different figures 393 in the treatment region 357 as illustrated in FIG. 3C. Contrast enhancing agents can be applied using stamps, rollers, sprays, stencils, or with agent-soaked gauze pads.

Following treatment, the contrast enhancing agent that remains typically has an undesirable appearance, particularly when it strongly absorbs light in the visible spectrum (i.e. for wavelengths of about 350 nm to about 750 nm). A bleaching agent can be applied to reduce the absorption of the contrast enhancing agent in the visible spectrum and thus reduce the visibility of the contrast enhancing agent. The appropriate choice of bleaching agent will depend on the choice of contrast enhancing agent that is used.

FIG. 3 describes an embodiment of a method of the invention that includes the bleaching of an applied contrast enhancing agent. This method comprises three steps. The first step 301 is the step of applying a contrast enhancing agent to the skin. The second step 302 is the step of delivering optical energy to the selected region of skin using a handpiece that measures one or more positional parameters of the handpiece and controlling the delivery of the optical energy in response to the measurement of the one or more positional parameters. The third step 303 is the step of applying bleaching agent to the skin to reduce the visibility of the contrast enhancing agent. Additional steps and/or substeps can be incorporated into this method. For example, additional steps between 301 and 303 may further comprise the steps of optically measuring one or more positional parameters of the handpiece and using the measured positional parameters to control the treatment parameters of the delivered optical energy.

In addition to the contrast enhancing agents described above, triaryl methane coloring agents (also known as the triphenal methane family of dyes) can be used as contrast enhancing agents. For example, Royal Blue fountain pen ink (CAS # 28983-56-4; C. Josef Lamy GmbH, Heidelberg, Germany; and National Ink, LLC, Santee, Calif.) is a non-toxic contrast enhancing agent that comprises one or more triphenyl methane coloring agents. Other examples of triaryl methane coloring dyes include Basic Blue 8 (Victoria Blue 4R), Basic Blue 15 (Night Blue), Basic Blue 20 (Methyl Green), Basic Blue 26 (Victoria Blue B), and other non-blue dyes such as Fuchsin, Crystal Violet, Fluorescein, Eosin, and Phenolphthalein. Basic Blue 15 is a common ingredient in food colorings and so is generally regarded as non-toxic.

A common chemical property of triaryl methane dyes is their central, unsaturated carbon atom. For these dyes, the outer orbitals of the central carbon atom are typically in the sp² configuration, which allows one of the pi electrons to become delocalized and to travel through the “free” p-orbitals of a chain of sp² hybridized carbon atoms. The electron energy levels of these delocalized electrons are affected by the spatial electrical potential profile of the chain of atoms. Converting the hybridization state for one or more of the sp² hybridized carbon atoms to sp³ will localize an electron and will block transmission of other electrons past the sp³ bond. Thus, the absorption spectrum of the contrast enhancing agent is changed by the change in hybridization state of the electrons that surround one or more central carbon atoms.

There are several chemical reactions that can be used to convert the sp² hybridized carbon atoms to sp³. For example, selected reducing agents can cause anions, for example perhydroxyl ions (OOH⁻) or hydrogen sulfite ions (HSO₃⁻), to bind to the sp² hybridized carbon atom. The attachment of one or more anions to the carbon atom can change the bond geometry to desirably confine the formerly free electrons. Spatial confinement of one or more electrons changes the separation between energy levels and thus the wavelengths of light that are absorbed. This shift in absorption spectrum typically occurs by increasing the energy gap between energy levels (i.e. the absorption spectrum is blue-shifted).

Thus, appropriate bleaching agents for many of the triaryl methane dyes are agents which produce anions, such as perhydroxyl or hydrogen sulfite ions. Examples of appropriate bleaching agents are included in the categories of peroxygen compounds, hypochlorites, reducing agents, and chlorine dioxide. Other examples of appropriate bleaching elements for use with selected contrast enhancing agents will be evident to those skilled in the art.

For each of these bleaching agents, the pH of the bleaching agent can affect the reaction rate significantly. For most bleaching agents, there is an optimal range of pH. For pH values below the desired range, there are not enough active anions to cause the bleaching reaction. For pH values above the desired range, the active anions can decompose or can cause damage to the skin.

For example, for peroxygen compounds and hypochlorites, the solution should be basic, preferably with a pH of about 8 to about 12, and more preferably with a pH of about 9 to about 10.5. The narrower range of pH is preferred because this range typically has a faster rate for the bleaching reaction. The pH of 9-10.5 is also desirable because it is typical of soaps and so is generally considered to be safe for application to the skin. A pH lower than 8, such as a pH of 7.0, can also work, but the bleaching action takes much longer to remove the color from the contrast enhancing agent.

As a specific example, the active bleaching species in hydrogen peroxide is the perhydroxyl ion, OOH⁻, which is formed through the ionization of H₂O₂ in water (H₂O₂+H₂O→H₃O⁺+OOH⁻). For hydrogen peroxide, which has an acid ionization constant of Kₐ=2*10⁻¹², pH values above about 9.0 are desirable to permit the ionization of hydrogen peroxide (H₂O₂) in water to form a significant concentration of perhydroxyl ions. However, when the pH exceeds about 11.0, the decomposition of perhydroxyl ions into hydroxide ions (OH⁻) and molecular oxygen accelerates. Hydroxide ions have a significantly slower bleaching action than perhydroxyl ions. So it is desirable to use hydrogen peroxide in water with a pH of between about 9.0 and about 11.0, and preferably between about 9.0 and about 10.5. The pH of the hydrogen peroxide and water solution can be adjusted by adding a builder such as sodium carbonate, ammonia, or sodium hydroxide that makes the solution more basic. Similar preferred pH ranges are applicable for other peroxygen compounds.

For some bleaching agents, it can be important that the bleaching agent is stabilized. For example, at room temperature, hydrogen peroxide decomposes very slowly into water and oxygen. The presence of certain cations (e.g. Fe³⁺, Mn²⁺, and Cu²⁺) can accelerate the decomposition. Therefore, hydrogen peroxide can be preferentially stabilized with complexing agents that sequester transition metal cations. Other bleaching agents, such as peroxygen compounds, can be similarly stabilized with complexing agents. Stabilization with complexing agents can desirably increase the shelf life of a bleaching agent.

For some bleaching agents, the bleaching action can be beneficially accelerated by adding an accelerating compound, such as tetra acetyl ethylene diamine. This is particularly true for peroxygen compounds. Other accelerating chemicals will be obvious to those skilled in the art. The choice of accelerating agent can be made based on the amount of contrast dye to be bleached, the desired concentration of bleaching agent, the desired bleaching rate, and the at acceptable bleaching temperature.

The bleaching action can also be accelerated for some bleaching agents by applying heat, for example by applying a heating pad or shining a bright lamp on the skin to heat the skin above normal temperature while the bleaching is performed.

Each of the bleaching agents mentioned above has particular advantages. For example, peroxygen compounds release perhydroxyl ions that are useful for bleaching and these bleaching agents are considered to be less damaging to the environment than other types of bleaching agents, such as hypochlorite based bleaches. Peroxygen compounds are also less irritating when applied to skin, particularly if the skin has open wounds. Examples of peroxygen compounds are hydrogen peroxide, benzoyl peroxide, and sodium percarbonate. Benzoyl peroxide is a particularly useful bleaching agent because it is commercially available in a cream form that can be easily applied to the skin and it is FDA approved for safe use on the skin.

Hypochlorite type bleaching agents release hypochlorite ions (OCl⁻) that are useful for bleaching and are typically faster acting than other types of bleaching agents and may be more effective for bleaching selected types of contrast enhancing agents. Hypochlorite type bleaching agents can be more irritating to the skin; so the concentration and pH of the bleaching agent should be carefully controlled. Examples of hypochlorite type bleaching agents are sodium hypochlorite (NaOCl) and calcium hypochlorite (Ca(OCl)₂).

Reducing agents can also be used as bleaching agents when paired with an appropriate contrast enhancing agent. For example, the absorption properties of some triaryl methane dyes can be altered by applying reducing agents that convert the double bonds of one or more central carbon atoms to single bonds. Some examples of reducing agents are sulfites (for example, sodium sulfite (Na₂SO₃), sodium hydrogen sulfite (NaHSO₃), and potassium sulfite (K₂SO₃)), bisulfites, dithionites (for example, sodium dithionite (Na₂S₂O₄)), sodium borohydride, and carbonites (for example, sodium carbonate (Na₂CO₃) and sodium hydrogencarbonate (NaHCO₃)). CO₂ that occurs in the atmosphere plus water forms carbonic acid, which can react with the hydroxide ions to reduce the efficacy of the bleaching action. To reduce this degradation, ethanol can be added. The addition of ethanol can be particularly effective for sulfites or dithionites.

Chlorine dioxide (ClO₂) is a fourth type of bleaching agent. This bleaching agent is safer than hypochlorite bleaches and doesn't produce dioxins which can be toxic or carcinogenic to humans.

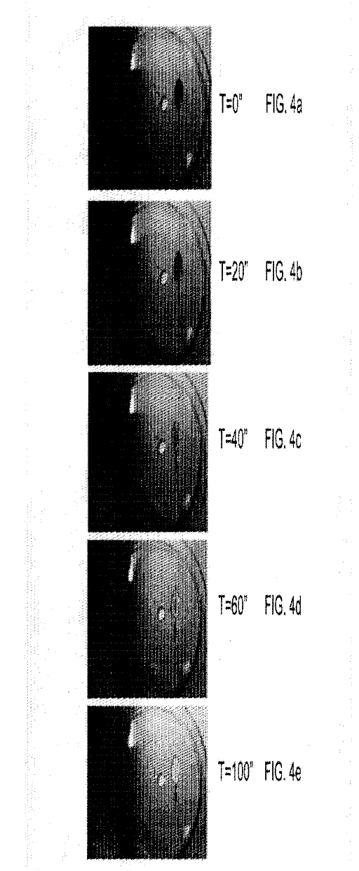

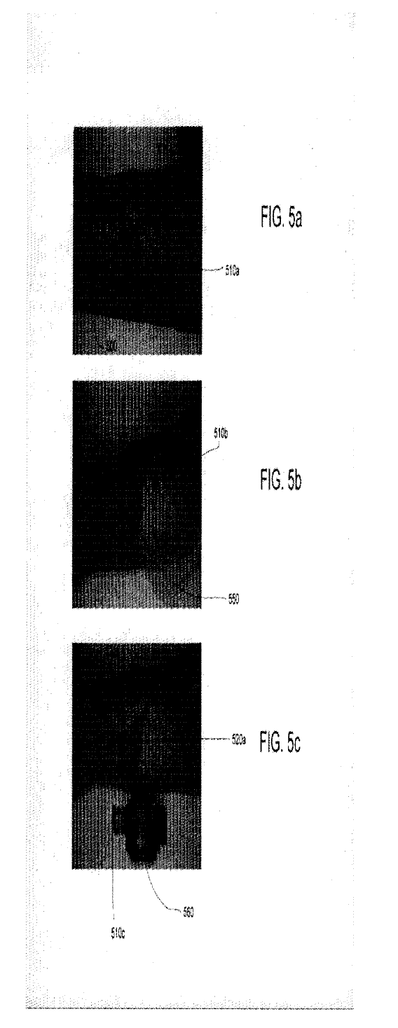

FIG. 4 shows a picture of one example of a bleaching agent that was used with a selected contrast enhancing agent. A drop of contrast enhancing agent (FD&C Blue #1 dye diluted in water with a concentration of 0.4% by mass) was placed into a Petri dish. A drop of bleaching solution comprising 3% H₂O₂, 2% NaOH, and water was added to the contrast enhancing agent. The bleaching agent had a pH of about 10. FIG. 4A shows the Petri dish immediately after adding the bleaching agent (before the color had changed significantly). FIGS. 4B, 4C, 4D, and 4E show the progression of the bleaching action 20, 40, 60, and 100 seconds after adding the bleaching agent, respectively. The visibility of the contrast enhancing agent is significantly reduced by the application of the bleaching agent due to the reduction of the optical absorption of the FD&C Blue #1 dye in the visible spectrum.

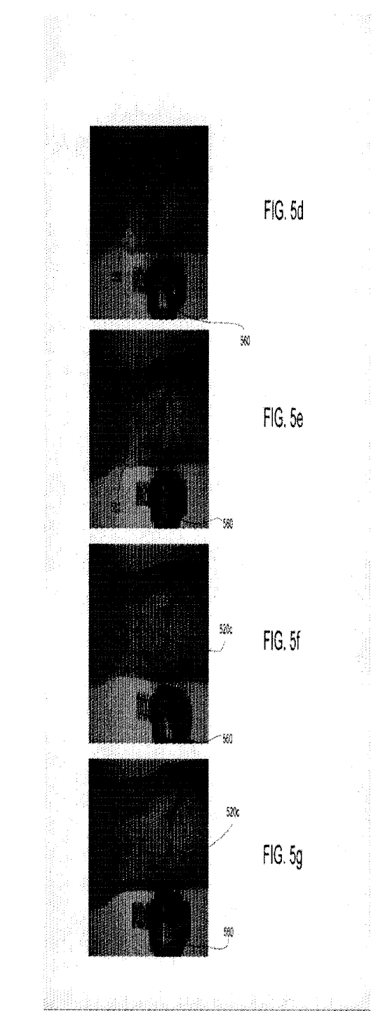

FIG. 5 shows a series of pictures of an example of a bleaching agent used with a contrast enhancing agent on human skin. A water-based mixture of FD&C Blue #1 in 4% concentration was applied onto human skin. A water-based contrast enhancing agent containing 3% hydrogen peroxide and 0.5% sodium hydroxide was applied onto a gauze pad until thoroughly wetted. The wetted gauze pad was laid on the blue skin, whereby the contrast enhancing blue dye was bleached within less than 1 minute.

Examples of combinations that have been successfully tested in a Petri dish are described in Table 1. In each of these cases, the visibility of the contrast enhancing agent in the right column was reduced significantly following application of the corresponding bleaching agent in the left column. This reduction in visibility occurred due to a significant reduction of the absorption of the contrast enhancing agent for one or more wavelengths in the visible spectrum.

TABLE 1

| Bleaching agent | Contrast enhancing agent |
|---|---|
| Na₂SO₃ (1.25%) | water blue |
| | FD&C Red 40(color change |
| | to orange) |
| | FD&C Yellow 6 (color |
| | change orange to yellow) |
| NaOH (5%) | water blue |
| H₂O_{2 (3%)} | water blue |
| | food blue |
| | FD&C Blue No. 1 |
| NaOH + H₂O₂ | water blue |
| 0.5% and 3% respectively, | food blue |
| (pH 10.6) | FD&C Blue No. 1 |
| | Pat. Blue Ca. |
| | Pat Blue Na. |
| | Pat Blue VF |
| | food green |
| | FD&C Yellow 6 |
| NaHCO₃ | water blue |
| NaOCl (0.6%, pH 9) | water blue |
| | food blue |
| | FD&C Blue No. 1 |
| | Pat. Blue Ca. |
| | Pat Blue Na. |
| | Pat Blue VF |
| | food green |
| | food red |
| | DC Red#33 |
| | DC Red#22 |
| | FD&C Red 40 |
| | DC Green Lake |
| | DC Blue Lake |
| | FD&C Yello 6 |
| | FD&C Yellow 5 |

All contrast enhancing agents described in Table 1 were applied onto white paper and left to dry. The contrast enhancing agents were then bleached using the chemicals in the concentrations listed in the left column of Table 1. All dyes bleached within 10 minutes. The concentrations listed in Table 1 describe concentrations of active agents in water based solutions.

The list of bleaching agents, concentrations, pH values, and contrast agents listed in Table 1 is not exhaustive. Other concentrations and pH and other combinations of bleaching agents and contrast agents are applicable.

In Table 1, Water Blue is also known as Methyl Blue (CAS #28983-56-4) and is less stable than many other dyes, which can make it easier to bleach with many bleaching agents. Solid peroxygen compounds dissolved in water can be used as bleaching agents for Water Blue. Pat. Blue Ca. is patent blue calcium salt (CAS #3536-49-0). Pat. Blue Na. is patent blue sodium (CAS #20262-76-4). Pat. Blue VF is patent blue VF (CAS #129-17-9). Food blue, food yellow, food green, and food red are commercially available food colorings (McCormick and Company, Inc., Sparks, Md.). FD&C Blue #1 is a blue dye that is FDA approved for use in cosmetic applications. These dyes were bleached with different bleaching agents as described in Table 1.

Note that the CAS numbers given above refer to the reference numbers for the chemicals in the database of chemical substances that is maintained by the Chemical Abstracts Service (Chemical Abstracts Service, Columbus, Ohio), which is a division of the American Chemical Society. Each substance in the database is identified by a unique CAS registry number.

Reducing agents that produce sulfites (SO₃²⁻) can be used as bleaching agents with Royal Blue ink. For example, solutions containing sodium sulfite (Na₂SO₃), sodium hydrogen sulfite (NaHSO₃), potassium sulfite (K₂SO₃), or combinations thereof could be used. Sodium sulfite solutions have particular advantages in that they have antibacterial properties.

Although the detailed description contains many specifics, these should not be construed as limiting the scope of the invention but merely as illustrating different examples and aspects of the invention. It should be appreciated that the scope of the invention includes other embodiments not discussed in detail above. For example, other bleaching agents can be used that will be evident to those skilled in the art. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. A method of dermatological treatment comprising the steps of:
applying a contrast enhancing agent to a selected region of skin;
manually manipulating a handpiece to effect delivery of electromagnetic treatment energy to cause a dermatological treatment of the selected region of skin;
measuring by a sensor at least one positional parameter of the handpiece, wherein the measurement by the sensor is enhanced by the contrast enhancing agent;
controlling the delivery of the electromagnetic treatment energy based on the at least one positional parameter; and
applying a bleaching agent to the selected region to reduce the visibility of the contrast enhancing agent in the visible spectrum.

2. The method of claim 1, wherein the step of measuring the at least one positional parameter comprises optically sensing the at least one positional parameter of the handpiece.

3. The method of claim 1, wherein the step of measuring the at least one positional parameter comprises optically sensing the at least one positional parameter of the handpiece using an optical mouse chip.

4. The method of claim 1, wherein the step of applying the contrast enhancing agent comprises applying the contrast enhancing agent topically to the selected region.

5. The method of claim 1, wherein the step of applying the contrast enhancing agent comprises disposing lines and/or patterns on the selected region.

6. The method of claim 1, wherein the step of applying the contrast enhancing agent comprises disposing equally spaced indicia on the selected region.

7. The method of claim 1, wherein the step of applying the contrast enhancing agent comprises disposing the contrast enhancing agent in a substantially uniform concentration that selectively decorates at least some wrinkles and pores.

8. The method of claim 1, wherein the contrast enhancing agent comprises a triphenyl methane compound.

9. The method of claim 1, wherein the contrast enhancing agent comprises at least one of methyl blue, water blue, aniline blue, royal blue, and basic blue 15.

10. The method of claim 1, wherein the contrast enhancing agent comprises at least one of basic blue 8, basic blue 20, basic blue 26, fuchsin, crystal violet, eosin, and phenolphthalein.

11. The method of claim 1, wherein the bleaching agent comprises at least one of a peroxygen compound, an oxidizing agent, a hypochlorite, a reducing agent, and chlorine dioxide.

12. The method of claim 11, wherein the bleaching agent comprises a peroxygen compound with a concentration in the range of about 3% by mass to about 20% by mass.

13. The method of claim 11, wherein the bleaching agent comprises a hypochlorite with a concentration in the range of about 0.1% by mass to about 3% by mass.

14. The method of claim 11, wherein the bleaching agent comprises a reducing agent with a concentration in the range of about 0.6% by mass to about 3% by mass.

15. The method of claim 11, wherein the bleaching agent comprises chlorine dioxide.

16. The method of claim 11, wherein the pH of the bleaching agent is between about 8.0 and about 12.0.

17. The method of claim 12, wherein the pH of the bleaching agent is between about 9.0 and about 11.0.

18. The method of claim 11, wherein the bleaching agent further comprises an accelerating agent.

19. The method of claim 18, wherein the accelerating agent comprises acetyl ethylene diamine.

20. The method of claim 11, wherein the contrast enhancing agent is FD&C Blue #1 and the bleaching agent comprises a peroxygen compound.

21. The method of claim 20, wherein the bleaching agent further comprises acetyl ethylene diamine.

22. The method of claim 1, wherein the bleaching agent is dispensed during treatment in a manner that indicates where treatment has been performed.

23. The method of claim 22, wherein the step of applying a bleaching agent comprises dispensing the bleaching agent from a disposable tip of the handpiece.

24. A manually manipulatable handpiece for effecting delivery of electromagnetic treatment energy to cause a dermatological treatment of the selected region of skin, the handpiece further comprising:
a sensor that measures at least one positional parameter of the handpiece, wherein the measurement by the sensor is enhanced by a contrast enhancing agent applied to the selected region of skin; and
a disposable tip that dispenses a bleaching agent to the selected region to reduce the visibility of the contrast enhancing agent in the visible spectrum, in a manner that indicates where treatment has been performed.
